(19) United States
(12) Patent Application Publication
(10) Pub. No.: US 2009/0048507 A1
(43) Pub. Date: Feb. 19, 2009

(54) Method and apparatus for imaging functional and electrical activities of the brain

(76) Inventors: Thorsten Feiweier, Poxdorf (DE); Diana Martin, Herzogenaurach (DE); Gunther Platsch, Rothenbach (DE); Sebastian Schmidt, Weisendorf (DE); Kristin Schmiedehausen, Palo Alto, CA (US); Michael Szimtenings, Bonn (DE)

Correspondence Address: HARNESS, DICKEY & PIERCE, P.L.C., P.O.BOX 8910, RESTON, VA, 20195, US

(21) Appl. No.: 12/222,210

(22) Filed: Aug. 5, 2008

(30) Foreign Application Priority Data
Aug. 7, 2007 (DE) 10 2007 037 103.0

(51) Int. Cl. A61B 5/055; A61B 5/0476; G01V 3/00; A61B 6/00

(52) U.S. Cl. 600/411; 600/544; 324/316; 324/307; 378/4

(57) ABSTRACT

A method and an apparatus for imaging functional and electrical activities of the brain are disclosed. In order to allow improved relative positional determination of the epileptogenic focus with reference to the EEG electrodes, a positron emission tomography measurement using at least one radiation detector, and a magnetic resonance imaging measurement using at least one coil for generating a basic magnetic field, at least one gradient coil and a radio-frequency antenna device are undertaken in at least one embodiment. In addition, in at least one embodiment an electroencephalography measurement using a plurality of electrodes for acquiring spatial and temporal changes of the electrical activities of the brain and a computed tomography measurement using at least one x-ray source and at least one x-ray detector are carried out. By way of an evaluation apparatus in at least one embodiment, a spatial correlation between the computed tomography measurement and the magnetic resonance imaging measurement is undertaken, so as to result in registration between the electroencephalography measurement and the positron emission tomography measurement.

### PRIORITY STATEMENT

The present application hereby claims priority under 35 U.S.C. §119 on German patent application number DE 10 2007 037 103.0 filed Aug. 7, 2007, the entire contents of which is hereby incorporated herein by reference.

### FIELD

Embodiments of the invention generally relate to a method and/or an apparatus for imaging functional and electrical activities of the brain.

### BACKGROUND

In preoperative epilepsy diagnostics, a multiplicity of different—in part imaging—methods is used to localize the epileptogenic focus and to assess the operability. These include magnetic resonance imaging (MRI), computed tomography (CT), single photon emission computed tomography (SPECT), and positron emission tomography (PET). In addition, brainwaves can be measured as a parameter of the activity of the neurons by means of surface and invasive electroencephalography (EEG) and magnetoencephalography (MEG). The examination results of the individual measurements are not sufficiently significant on their own. Only correlation and combined appraisal of the results affords sufficient accuracy.

In the case of a surface EEG, electrodes are affixed to the scalp and potential differences between the individual electrodes are recorded. The distance of the electrodes from the brain surface is comparatively large, so that the measured signal is relatively weak and additionally superposed by signals from other areas of the brain. As a result of this, the anatomical association of the measured signals with a structure of the brain is difficult. More accurate information is obtained only with electrodes sitting directly on the surface of the brain. Such an invasive EEG itself is not an imaging method in the proper sense: individual electrodes, strip electrodes or plate electrodes are applied to the surface of the brain by the neurosurgeon during an operation, or long electrodes are pushed up against the brain through holes located in the base of the skull. Subsequently, the patient is observed for a few days with the video EEG in order to obtain an ictal (during a fit) EEG during epileptic fits. Using this, an epileptogenic focus can be delimited in a substantially more accurate manner than with the surface EEG, and substantially more precise localization of the focus is possible.

However, in the case of both a surface EEG and an invasive EEG, the exact spatial coordinates of the epileptogenic focus, by means of which a neurosurgeon could plan a procedure, are missing. Therefore, the registration of EEG measurements or functional measurements, such as PET or SPECT, to MRI, results would be desirable. However, due to the metal content of the EEG electrodes fitted during an operation, no MRI can be carried out during the period in which the electrodes are fitted. PET does not provide a remedy since the positions of the electrodes cannot be recognized using it. It follows that the coordinates of the electrodes must be determined differently. The determination of the coordinates is possible using computed tomography (CT).

The prior art discloses hybrid modalities, such as PET/CT or MRI/PET systems, in which a plurality of imaging methods are combined in one system. A combined MRI/PET system permits, for example, simultaneous and isocentric acquisition of MRI and PET data. The functional methods are in addition often used to support the findings from the aforementioned EEG measurements. However, functional methods, such as PET and SPECT, display anatomical structures with only limited accuracy, which in addition depends on the tracers used as well.

MRI/PET is preferable to PET/CT for a number of reasons: operation planning is carried out on the basis of the MRI since this produces the best anatomical resolution and details can be recognized very well using it. The CT component of the PET/CT does not satisfy these requirements.

However, even if the exact position of the electrodes in a PET image could be specified using a PET/CT, correlation of the PET with the EEG is not possible, since, during the period in which invasive electrodes are fitted, a PET can be afflicted with artifacts: absorption effects occur due to the metal plates, irritated states of the brain and meninges due to the surgical procedure and the electrodes can lead to non-physiological storage patterns in the PET and mean that it cannot be assessed, or assessed only with difficulty. Thus the correlation between invasive EEG and PET would be subject to major uncertainty. For this reason, exact correlation between electrode positioning (and thus pathological electrical activity) and the changes (increased or decreased metabolism, decreased receptor density) is not possible in PET, and increases the uncertainty in operation planning.

For this reason, the PET/CT would require a further CT during the period in which the electrodes are fitted. This would then have to be correlated with the PET/CT, and the PET/CT would then have to be correlated with the MRI. Overall this procedure would lead to a higher radiation exposure, since two CT records would have to be made. Furthermore, the registration complexity would be increased, since the EEG would have to be correlated with the CT and with the MRI via the PET/CT.

### SUMMARY

In at least one embodiment of the present invention, a method and/or an apparatus are specified, which allow improved relative positional determination of the epileptogenic focus with reference to the EEG electrodes.

By way of simultaneous and isocentric recording of PET and MRI by the MRI/PET scanner, the PET is exactly registered to the MRI. The MRI can then be registered to the CT without problems, since significantly more anatomical details are displayed. As a result, the PET is automatically registered to the CT, and thus also to the electrode position. By these, it is possible to then compare the functional results of the invasive EEG exactly with the results of the PET. According to at least one embodiment of the invention, this exploits the fact that the isocentric and simultaneous recording of MRI and PET, allows all data of the third and (indirectly) fourth modalities, which until now only were registered to the MRI, thus to be exactly registered to the PET. This then permits findings with an overview over all the modalities involved.

The method according to at least one embodiment of the invention for imaging functional processes in the brain has the following steps: recording a positron emission tomography measurement using at least one radiation detector for recording positron annihilation radiation from an examination area, recording a magnetic resonance imaging measurement using at least one coil for generating a basic magnetic field, one gradient coil for generating a magnetic gradient field in the examination area and a radio-frequency antenna device for sending excitation pulses into the examination area and for receiving magnetic resonance signals from the examination area, wherein the positron emission tomography measurement and the magnetic resonance imaging measurement are carried out substantially simultaneously and isocentrically, so that this results in registration between the positron emission tomography measurement and the magnetic resonance imaging measurement, and recording an electroencephalography measurement using a plurality of electrodes for recording spatial and temporal changes of brainwaves, recording a computed tomography measurement using at least one x-ray source for generating x-ray radiation in the examination area and at least one x-ray detector for acquiring x-ray radiation originating from the examination area so that the electrodes of the EEG measurement can be identified, wherein the computed tomography measurement is carried out during a period in which the electrodes are fitted for the electroencephalography measurement, so that this results in registration between the electroencephalography measurement and the computed tomography measurement, and wherein, by means of an evaluation apparatus, a spatial correlation between the computed tomography measurement and the magnetic resonance imaging measurement is undertaken, so that this results in registration between the electroencephalography measurement and the positron emission tomography measurement.

In particular, the record of the positron emission tomography measurement, the record of the magnetic resonance imaging measurement, and the record of the electro-encephalography measurement are displayed simultaneously on a display device. This gives the treating medical practitioner a direct overview of all the information available about an area of the brain.

In a further example embodiment, this is followed by the following steps: determining first reference points in the record of the magnetic resonance imaging measurement, determining second reference points in the record of the computed tomography measurement, aligning the first and second reference points and displaying the record of the positron emission tomography measurement, the record of the magnetic resonance imaging measurement, and the record of the electroencephalography measurement on the display device so that the first and second reference points are substantially coincident. This ensures that all measurements are exactly associated with the respective anatomical areas.

In a further example embodiment, the spatial correlation between the computed tomography measurement and the magnetic resonance imaging measurement is undertaken by the evaluation apparatus on the basis of anatomical details. This is the simplest and most reliable manner of automating the correlation, so that manual moving of files is no longer necessary.

In a further example embodiment, bipolar and/or unipolar derivations of the EEG are displayed together with the PET data, the MRI data and the CT data. This allows the treating medical practitioner to examine the changes in the electrical activities of the brain in addition to the (functional) PET data and, in the process, rely on the anatomical relationships in the affected area of the brain, or take these into consideration.

Accordingly, the apparatus according to at least one embodiment of the invention for imaging functional processes in the brain is provided with: at least one radiation detector for recording positron annihilation radiation from an examination area as a record of a positron emission tomography measurement, at least one coil for generating a basic magnetic field, one gradient coil for generating a magnetic gradient field in the examination area and a radio-frequency antenna device for sending excitation pulses into the examination area and for receiving magnetic resonance signals from the examination area as a record of a magnetic resonance imaging measurement, wherein the positron emission tomography measurement and the magnetic resonance imaging measurement are substantially carried out simultaneously and isocentrically, so that this results in registration between the positron emission tomography measurement and the magnetic resonance imaging measurement, and a plurality of electrodes for recording spatial and temporal changes of the electrical activities of the brain as a record of an invasive electroencephalography measurement, at least one x-ray source for generating x-ray radiation in the examination area and at least one x-ray detector for recording x-ray radiation originating from the examination area as a record of a computed tomography measurement, so that the electrodes for the EEG measurement can be identified, wherein, by means of an evaluation apparatus, a spatial correlation between the computed tomography measurement and the magnetic resonance imaging measurement is undertaken, so that this results in registration between the electroencephalography measurement and the positron emission tomography measurement.

Preferably, the radiation detector and the at least one gradient coil are arranged coaxially and substantially at the same axial height around the examination area in the apparatus. By means of this, MRI and PET data are recorded simultaneously and separate adaptation of the data is no longer required.

One advantage of the method according to at least one embodiment of the invention is that, in addition to functional data (from the PET), activity signals (from the EEG) are also available to the treating medical practitioner.

### BRIEF DESCRIPTION OF THE DRAWINGS

Further features and advantages of the invention emerge from the following description of exemplary embodiments, with reference being made to the attached drawings.

FIG. 1 shows a combined MRI/PET scanner according to the prior art in a perspective illustration.

FIG. 2 shows the combined MRI/PET scanner according to FIG. 1 in a cross section.

FIG. 3 schematically shows a CT record of the brain with simultaneously fitted invasive EEG electrodes.

FIG. 4 shows a schematic illustration of an embodiment according to the invention of the method or apparatus for imaging functional processes in the brain.

The drawings are not to scale. Identical elements, or elements with the same effect, are provided with the same reference symbols.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

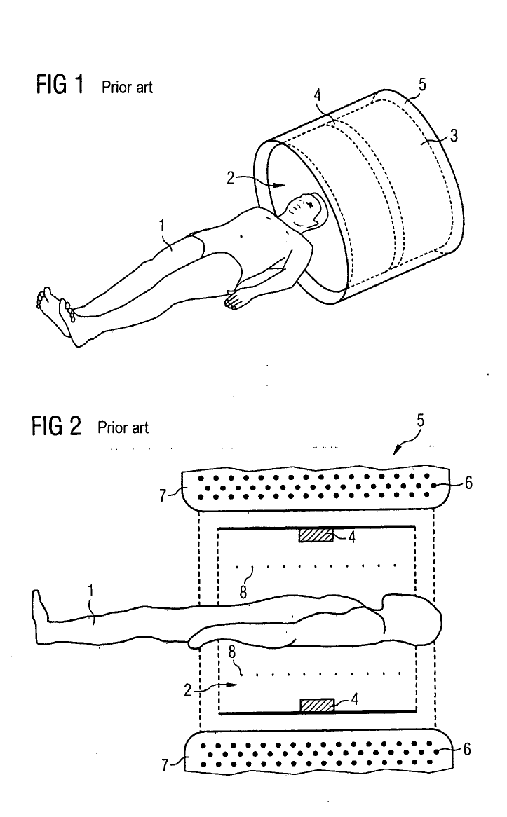

FIG. 1 illustrates a combination of positron emission tomography (PET) and magnetic resonance imaging (MRI). In a combined PET and MRI, a subject 1 is placed in an examination area 2. The examination area 2 is directly surrounded by a PET apparatus 3 which includes a detector device 4. In the detector device 4 of the PET apparatus 3, photons, which are created by the annihilation of a positron together with an electron, are detected. Positrons are released during radioactive decay of an isotope within the body of the subject 1. For this purpose, appropriate medicines or preparations (so-called radiopharmaceuticals) are administered to the subject prior to the examination, composed of a radioactive isotope which accumulates in the tissue according to the biological function. The positrons released with an initial energy of between 0 eV and a few MeV are scattered in the surrounding tissue and thus slowed down more and more. Once they have reached a particular kinetic energy, they can be captured by an electron and annihilate with the latter after 0.1 ns to 150 ns, with usually two 511 keV photons being emitted in propagation directions which are diametrically opposite to one another. The detector device 4 is in general an arrangement of scintillation crystals (not shown) which are arranged annularly around the examination area 2. In the scintillation crystals, the photons with an energy of 511 keV (annihilation radiation of the positrons) are converted to light quanta, which in turn are passed, preferably via optical waveguides (not shown), to photodetectors (not shown) which generate electrical output signals as a function of the number of light quanta.

So that it is possible to anatomically associate the examination results of the PET measurement in the subject 1, the PET apparatus is combined with an MRI apparatus 5. Both apparatuses are explained below on the basis of FIG. 2, which illustrates the design of a combined PET and MRI apparatus in a cross section. The examination area 2 of the combined PET/MRI apparatus is substantially defined by a gradient coil 6 in a housing 7 and a radio-frequency antenna device 8. The subject 1 is partly located in the examination area 2. The gradient coil 6 is arranged on the outside around the examination area 2 and generates a magnetic field in the examination area 2, the strength of which changes from left to right in the examination area. The gradient field serves for encoding spatial information.

The polarization of the spins of the nuclei of the subject 1 is produced by a coil (not shown) for the basic magnetic field B₀, and this coil for the basic magnetic field concentrically surrounds the gradient coil. By way of the basic magnetic field, the spins of the nuclei in the body of the subject 1 are at least partly aligned, so that the degeneracy of their magnetic quantum number is lifted. Transitions between the no-longer degenerate states are induced by the radio-frequency antenna device 8. The relaxation signals of the transitions are recorded by the same radio-frequency antenna device and transferred to processing electronics (not shown). Subsequently, they are displayed graphically for evaluation.

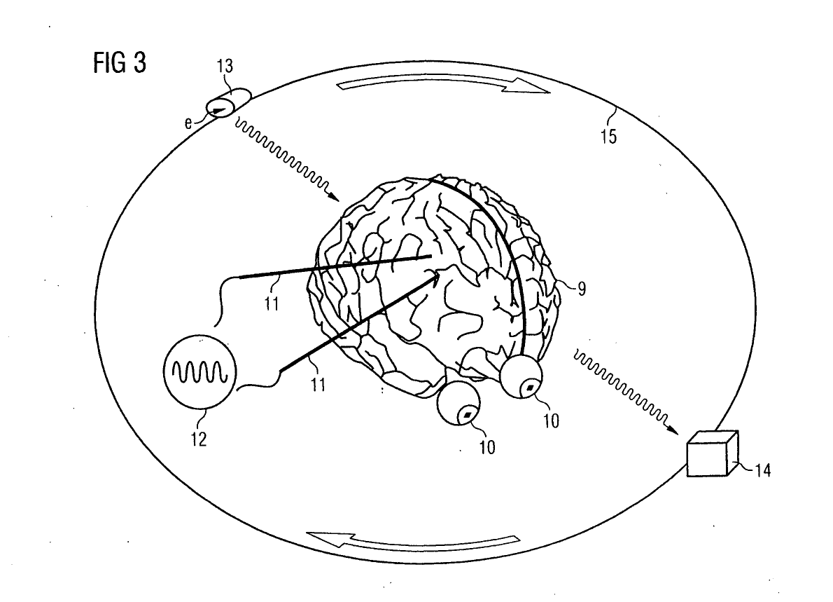

The basic principle of an invasive electroencephalography (EEG) measurement will be explained with reference to FIG. 3. In the case of an invasive EEG, a plurality of electrodes 11 are positioned on the surface of the brain 9. For the sake of clarity, only two electrodes are shown in FIG. 3; however, in principle, a plurality of electrodes can simultaneously be placed in the brain 9 and can also be present in the form of strip or plate electrodes with a plurality of individual electrodes. Electrical potentials of the brain 9 are recorded by the electrodes 11. In this case, bipolar signals can be derived, i.e. potential differences between pairs of electrodes, or the potential differences between the individual electrodes compared to an average of all electrodes 11 can be derived (unipolar derivation). A bipolar derivation 12 is shown in FIG. 3, indicating that the potential differences in general have a wavelike temporal profile.

According to an embodiment of the invention, a computed tomography measurement is carried out during the period in which the electrodes are fitted, which generally lasts for a few days—where necessary, it is also carried out simultaneously with the EEG measurement. For this purpose, the apparatus has an x-ray source 13, the anode of which is shown in FIG. 3. Electrons “e⁻” are incident on the anode 13 and slowed down. The desired bremsstrahlung is generated during the slowing of the electrons in the material of the anode. The bremsstrahlung, indicated as a wave train in FIG. 3, passes through the brain 9 of the subject 1 and is recorded on the opposite side by a detector 14, where its intensity is analyzed and processed. Depending on the attenuation of the x-ray radiation through the brain 9, more or less dense tissue is present.

In order to obtain a slice image of the respective plane of the brain on which the x-ray source 13 and x-ray detector 14 are located, the x-ray source 13 and the detector 14 together revolve around the subject 1, or the body part 9 of the subject 1 which is of interest, on a circular path 15 or on a helical path (not shown). The revolution is indicated in FIG. 3 by way of arrows. The intensity profile of the radiation arriving at the detector 14 is recorded in this case and reconstructed using an algorithm, which is not explained in any more detail here, to provide spatial information about the internal structure of the corresponding body part 9 of interest. The electrodes can be clearly identified in the CT record.

In the case of computed tomography, it is important to expose organs which are particularly sensitive to as little radiation as possible. This is relevant in particular for the eyes 10 of the subject. The plane of the x-ray examination must therefore be placed in such a way that the eyes are exposed to no or very little radiation. This can be carried out by displacing the plane in the vertical direction (with reference to the body axis of the subject 1) or by tilting the plane with respect to the body axis of the subject 1.

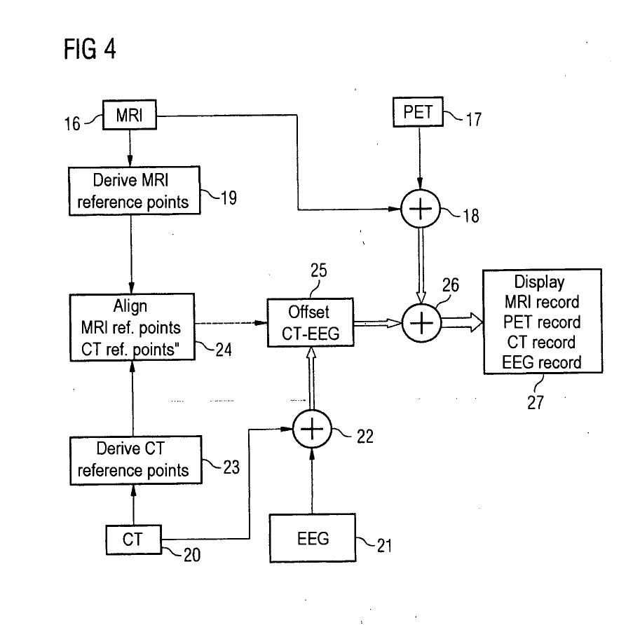

The results of a measurement using the electrodes 11 as sensors in FIG. 3, which are implanted in a brain 9, can be visualized as indicated in 12. In particular, it is always possible in the case of a plurality of electrode pairs 11 for a derivation 12 to be shown, or be superposed on a display, at those locations at which the respective electrodes are seated in the brain. This spatial information is obtained by joint evaluation of the CT data and the MRI data, as will be explained in more detail below on the basis of FIG. 4.

In an embodiment of the invention, all the available information is displayed at a glance to the treating medical practitioner, i.e. the anatomical information from the MRI image and, if applicable, the CT image, and also the functional information from the PET image and in particular the electrical information from the EEG, by means of which very rapidly changing parameters can be displayed.

The method according to an embodiment of the invention and the apparatus according to an embodiment of the invention are explained in the following text on the basis of FIG. 4.

In step 16, or with the apparatus 16, an MRI measurement is recorded. Simultaneously and at the same location in the examination area 2, a PET measurement is recorded in step 17, or with the apparatus 17. Both measurements are combined or superposed on one another at 18, so that they can be displayed simultaneously on one and the same display. This allows the observer to associate functional results from the PET with anatomical results of the MRI.

According to an embodiment of the invention, at 19, at least three spatial reference points are determined in the MRI record, independently of the PET measurement and its superposition with the MRI measurement.

At 20, a CT measurement is recorded, which likewise permits a statement about the anatomical conditions but does not require a magnetic field. Analogously to 19, at least three spatial reference points are determined in the CT record at 23. Furthermore, a combination of the CT measurement 20 and the EEG measurement 21 is created in the superposition 22. In this manner, a spatial association in the brain 9 is also obtained for the EEG.

The two (or more) reference points from 19 and 23 are compared with one another at 24, and depending on this comparison, an appropriate value is calculated which is intended to allow the records to be combined in the further stages of the method. The details of this comparison are very complex, since the CT recording and the MRI recording can neither be undertaken at the same location, i.e. under the same geometrical conditions for the subject, nor at the same time. Thus, the reference points in the two records have to first of all be defined independently from one another. In the two records, points are preferably selected which can also be identified easily and at an exact location in the respective other record. In particular, particularly striking anatomical features are suitable for this.

The results of the comparison from 24 are then used to combine the records from 16 and 20 in such a way that they are virtually coincident. For this purpose, it will generally be necessary to displace and rotate one of the records with respect to the other record. In the embodiment of the invention according to FIG. 4, the CT record is displaced in 25 so that it can be superposed on the MRI record in such a way that the chosen reference points are coincident in the two records. In the process, this displacement is also applied to the respective derivation 12 illustrated in FIG. 3.

As an alternative to registration by way of reference points, automatic registration, e.g. using the known “mutual information” algorithm, can also be undertaken. The registration can just as well be undertaken by the user.

At 26, the CT/EEG record from 22 is superposed on the MRI/PET record from 18. All information is now combined in one display, namely spatial information with a high resolution from the MRI record, first functional information with metabolic data from the PET record and second functional information with physical data (electrical distribution) from the EEG measurement in conjunction with the CT measurement. This combined information is finally displayed together at 27.

Thus, the exact correlation of a decreased glucose metabolism, decreased benzodiazepine receptor density, or an increased tryptophan uptake (in the case of tuberous sclerosis) in the PET with the detection of abnormal ictal and interictal electrical activity (during and in between fits) in the invasive EEG in a particular brain region increases its diagnostic reliability. The exact registration of the results of the two functional methods, which can detect an epileptogenic focus with greater confidence than anatomical methods, to the exact anatomical information of the MRI is a prerequisite for planning a surgical procedure and thus permits the neurosurgical removal of the epileptogenic focus. The anatomical guiding structures, which the neurosurgeon sees during the procedure and is able to identify, can be displayed only in the MRI, and the MRI serves as the basis for neuron navigation.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for imaging functional and electrical activities of the brain, comprising:
recording a positron emission tomography measurement using at least one radiation detector for recording positron annihilation radiation from an examination area;
recording a magnetic resonance imaging measurement using at least one coil for generating a basic magnetic field, at least one gradient coil for generating a magnetic gradient field in the examination area and a radio-frequency antenna device for sending excitation pulses into the examination area and for receiving magnetic resonance signals from the examination area, the positron emission tomography measurement and the magnetic resonance imaging measurement being carried out substantially simultaneously and isocentrically, so as to result in registration between the positron emission tomography measurement and the magnetic resonance imaging measurement;
recording an electroencephalography measurement using a plurality of electrodes for acquiring spatial and temporal changes of brainwaves;
recording a computed tomography measurement using at least one x-ray source for generating x-ray radiation in the examination area and at least one x-ray detector for acquiring x-ray radiation originating from the examination area so as to identify the electrodes of the EEG measurement, wherein the computed tomography measurement is carried out during a period in which electrodes are fitted for the electroencephalography measurement, so as to result in registration between the electroencephalography measurement and the computed tomography measurement; and
carrying out, via an evaluation apparatus, a spatial correlation between the computed tomography measurement and the magnetic resonance imaging measurement, so as to result in registration between the electroencephalography measurement and the positron emission tomography measurement.

2. The method as claimed in claim 1, further comprising:
displaying the record of the positron emission tomography measurement, the record of the magnetic resonance imaging measurement, and the record of the electroencephalography measurement, simultaneously on a display device.

3. The method as claimed in claim 2, further comprising:
determining first reference points in the record of the magnetic resonance imaging measurement;
determining second reference points in the record of the computed tomography measurement;
aligning the first and second reference points and displaying the record of the positron emission tomography measurement, the record of the magnetic resonance imaging measurement, and the record of the electroencephalography measurement on the display device so that the first and second reference points are substantially coincident.

4. The method as claimed in claim 3, wherein the spatial correlation between the computed tomography measurement and the magnetic resonance imaging measurement is undertaken by the evaluation apparatus on the basis of anatomical details.

5. The method as claimed in claim 1, wherein at least one of bipolar and unipolar derivations of the EEG are displayed together with the PET data, the MRI data and the CT data.

6. An apparatus for imaging functional and electrical activities of the brain, comprising:
at least one radiation detector to record positron annihilation radiation from an examination area as a record of a positron emission tomography measurement;
at least one coil to generate a basic magnetic field;
at least one gradient coil to generate a magnetic gradient field in the examination area;
a radio-frequency antenna device to send excitation pulses into the examination area and to receive magnetic resonance signals from the examination area as a record of a magnetic resonance imaging measurement, the positron emission tomography measurement and the magnetic resonance imaging measurement being carried out substantially simultaneously and isocentrically, so to result in registration between the positron emission tomography measurement and the magnetic resonance imaging measurement;
a plurality of electrodes to record spatial and temporal changes of the electrical activities of the brain as a record of an invasive electroencephalography measurement;
at least one x-ray source to generate x-ray radiation in the examination area; and
at least one x-ray detector to record x-ray radiation originating from the examination area as a record of a computed tomography measurement, so that the electrodes for the EEG measurement are identifiable, wherein, by way of an evaluation apparatus, a spatial correlation between the computed tomography measurement and the magnetic resonance imaging measurement is undertaken, so as to result in registration between the electroencephalography measurement and the positron emission tomography measurement.

7. The apparatus as claimed in claim 6, wherein the radiation detector and the at least one gradient coil are arranged coaxially and substantially at the same axial height around the examination area.

8. The method as claimed in claim 2, wherein at least one of bipolar and unipolar derivations of the EEG are displayed together with the PET data, the MRI data and the CT data.

9. The method as claimed in claim 3, wherein at least one of bipolar and unipolar derivations of the EEG are displayed together with the PET data, the MRI data and the CT data.

10. The method as claimed in claim 4, wherein at least one of bipolar and unipolar derivations of the EEG are displayed together with the PET data, the MRI data and the CT data.

11. A computer readable medium including program segments for, when executed on a computer device, causing the computer device to implement the method of claim 1.

12. The apparatus as claimed in claim 6, wherein the apparatus further comprises the evaluation apparatus.

13. An apparatus for imaging functional and electrical activities of the brain, comprising:
means for recording a positron emission tomography measurement by recording positron annihilation radiation from an examination area;
means for recording a magnetic resonance imaging measurement by generating a basic magnetic field, by generating a magnetic gradient field in the examination area and by sending excitation pulses into the examination area and receiving magnetic resonance signals from the examination area, the positron emission tomography measurement and the magnetic resonance imaging measurement being carried out substantially simultaneously and isocentrically, so as to result in registration between the positron emission tomography measurement and the magnetic resonance imaging measurement;
means for recording an electroencephalography measurement by acquiring spatial and temporal changes of brainwaves;
means for recording a computed tomography measurement by generating x-ray radiation in the examination area and by acquiring x-ray radiation originating from the examination area so as to identify the electrodes of the EEG measurement, wherein the computed tomography measurement is carried out during a period in which electrodes are fitted for the electroencephalography measurement, so as to result in registration between the electroencephalography measurement and the computed tomography measurement; and
means for carrying out a spatial correlation between the computed tomography measurement and the magnetic resonance imaging measurement, so as to result in registration between the electroencephalography measurement and the positron emission tomography measurement.
